(19) United States
(12) Patent Application Publication
(10) Pub. No.: US 2018/0015223 A1
(43) Pub. Date: Jan. 18, 2018

(54) Multi-Use Injection System

(71) Applicant: MEDAXOR PTY LTD, Scoresby, Victoria (AU)

(72) Inventor: Andreas AESCHLIMANN, Scoresby (AU)

(21) Appl. No.: 15/528,097

(22) PCT Filed: Dec. 18, 2015
(86) PCT No.: PCT/IB2015/059754

§ 371 Date: May 18, 2017

(30) Foreign Application Priority Data
Dec. 19, 2014 (AU) 2014905150

(51) Int. Cl. A61M 5/20; A61M 5/24; A61M 5/315; A61M 5/32; G06F 19/00

(52) U.S. Cl. CPC A61M 5/20; A61M 5/31553; A61M 5/31596; A61M 5/3204; A61M 5/24; A61M 2205/3553; A61M 2005/2013; A61M 2205/50; A61M 2205/502; A61M 2205/8206; G06F 19/3468; A61M 2202/04

(57) ABSTRACT

The present invention provides an injection pen system comprising of a driver assembly for a drug container; a syringe assembly; a power source for generating electrical energy; electrical communication means in each of the driver assembly and syringe assembly wherein the driver assembly and syringe assembly are in electrical communication; and at least one microprocessor programmed to monitor electrical signals produced by the injection pen components. The electrical signals may be produced at predefined intervals and they may be wirelessly transmitted. The transmitted signals may trigger commands within a computer program monitoring the injection process. The computer program may be spaced from the injection and driver assemblies. The injection pen system may incorporate a two-way switch activated by a trigger initiated by pulling a cap from a primary drug container or pushing the drug container adjacent a subject. The activation may cause the mixing of a lyophilised substrate with a fluid for injection.

### TECHNICAL FIELD

The present invention relates to fluid delivery devices and systems, in particular, medical syringes, and more particularly, medical syringe pens and systems for self-injection.

### RELATED APPLICATION

This application claims the priority and benefit of Australian provisional application 2014905150 filed Dec. 19, 2014.

### BACKGROUND ART

Many pharmaceutically active substances must be delivered as injectable solutions using a hypodermic needle and syringe. In recent years, self-injecting systems or self-injection pens have become more popular. A shift to pre-filled, disposable, self-injection devices aims to address convenience, regulatory compliance requirements, and needle phobia/patient compliance. Such systems are rapidly expanding across therapeutic areas such as devices for the treatment of diabetes, multiple sclerosis (MS), rheumatoid arthritis (RA), fertility, osteoporosis, hepatitis, oncology, anaemia and migraine headaches. Type II diabetes is increasing world-wide and some therapies, like glucagon-like peptides, require diligent self-injection treatment programmes.

Traditionally, a self-injection pen is designed around a primary drug container in the form of a pre-filled syringe or cartridge. Self-injection devices such as “pen injectors” and “auto-injectors” act as the secondary packaging for the primary drug container (cartridges or prefilled syringes). The device is designed to simplify the drug administration process for the user. Some of the key characteristics are: usability, end-of-dose indication, automatically activated needle shields preventing sharps injuries, and visual indication that dose has been fully delivered. The pen injector is the interface between the primary drug container and the patient.

The primary drug container, prefilled syringe or cartridge, in which the substance is contained, is filled with automated aseptic filling lines. After filling and closing the primary container with the appropriate closure system there is a small amount of air contained in the primary drug container. This air has to be removed from the primary drug container by the user before an injection can be given.

A new user on a new treatment plan needs to be aware of the timing and amount of medicine to use. For example, a program involving daily, or even more frequently, injections of insulin are often necessary for diabetes sufferers. A user may also want to track additional health related data together with tracking treatment plan details. A user may also desire to minimise the chance that a needle stick injury might occur when handling a self-injection system. To eliminate the need for drawing up from a vial or reconstituting from two vials, medicine manufacturers often develop pre-filled syringes of medicines to help with this problem.

Pharmaceutical companies are also reformulating some substances in order to optimise the frequency of injection. This is particularly true in the case of non-chronic diseases, or when the treatment regimen dictates infrequent drug administration (e.g., once every 2 or 4 weeks). In such cases the user might be less familiar with the self-injection device and may have to refer to the instruction manual for use every time the user is due for an injection.

Existing self-injecting devices do not offer an integrated interface to digital health databases enabling automatic control of prescribed therapy regime or improved patient engagement with the digital health systems. In many cases end-of-dose delivery indication is cumbersome and generally a user or patient has to countdown to ensure a full dose has been delivered from the syringe.

Many self-injection devices require multiple administration steps and are therefore complicated to use. In particular, dual-chamber injection pens where mixing of lyophilised drug and diluent and priming is required can have multiple administration steps.

What is needed is a commercially viable and compact injection system for monitoring and recording multiple injections from a single pen and other health related data.

### SUMMARY OF INVENTION

In recent years wireless communication technology has become popular but it has failed to be used in self-injection devices possibly due to its cost, limitation in size and power consumption. Recently, mobile operating systems such as Android and iOS are able to support new and further developed wireless communication protocols such as Bluetooth low energy (BLE), Near Field Communication (NFC), and ANT. The present invention most advantageously exploits these technologies by utilising the screen of a telecommunication device as a digital and interactive instruction means for use with an injection system. Further, the invention incorporates a communication interface which enables the automated control and monitoring of a prescribed therapy regime and helps to improve patient engagement and provide online user feedback. Further still, the invention provides that a patient may record additional health data in the mobile application disposed on the mobile telecommunication device.

The present invention provides apparatus and systems for delivering aqueous fluids, in particular, medicines and other therapeutic fluids, by injection in multiple events over time in a treatment program. In one aspect, the invention provides a housing, a primary drug container, and a control system for enabling injection and monitoring the injection process. Alternatively the apparatus described herein may be used in combination with a variety of different primary drug containers. The invention provides an apparatus to generate signals at predefined stages of the injection process. The signal sequencing is used to trigger appropriate prompts and commands within the computer software application and enables the recording of details such as end of dose delivery etc, of an injection process.

In another aspect, the invention provides an injection pen system comprising of a driver assembly for a drug container; and a syringe assembly; a power source for generating electrical energy; electrical communication means in each of the driver assembly and syringe assembly wherein the driver assembly and syringe assembly are in electrical communication; and at least one microprocessor programmed to monitor electrical signals produced by the injection pen components. The injection pen system preferably may generate the electrical signals at predefined intervals. The electrical signals are preferably wirelessly transmitted. The transmitted signals preferably trigger commands within a computer program monitoring the injection process. Preferably the programmed microprocessor is spaced from the driver assembly and syringe assembly. Preferably the programmed microprocessor is incorporated into a mobile telecommunication device. Preferably the mobile telecommunications device incorporates a telephone and other functions. Preferably, the injection pen system incorporates a two-way switch which is preferably activated by a trigger. The trigger may incorporate a primary drug container having a cap or cover wherein the trigger activation occurs by removing the cap or cover. The trigger may be activated by pushing the primary drug container adjacent a subject, preferably on the skin. Preferably the activation causes mixing of a lyophilised substrate with a fluid for injection.

The invention incorporates a communication interface which enables the automated control and monitoring of a prescribed therapy regime and helps to improve patient engagement and provide online user feedback.

In another aspect the invention provides an apparatus to generate signals at predefined stages of the injection process. The signal sequencing, signals which may be wirelessly transmitted, is used to trigger appropriate prompts and commands within the app and allows to record details such as end of dose delivery or other parameters or steps in an injection process. In particular, the invention provides a two-way way switch in some embodiments for generating signals which are wirelessly transmitted to a computer software application, which may prompt and commands within the application. The two-way switch may be activated by removing a cap or cover from a primary drug container and which may push a primary drug container onto skin as a push-trigger, for example. The invention may include a means for drug mixing of lyophilised compounds. This mixing may be sub-routine which may be triggered by a two-way switch as a pull trigger

Preferably, the insertion of the primary drug container into the injection pen system automatically powers on the device.

In some embodiments, the invention provides a mechanism to automatically prime (expel air) from the primary drug container for an injection system and provide instant feedback to a user after priming is completed via the GUI of the mobile communication device.

The invention provides a method for utilising the screen of a mobile telecommunication device to provide feedback to the injection pen system user. For example, the user may notified via the screen of the mobile telecommunication device, such as a mobile phone, when mixing is completed, when injection is completed, when the device or system is powered on, when injection is initiated or other parameters or steps of the injection process.

The injection pen system may include a drug container cover which is not attached with a hinge but is instead guided into position by a rotational sliding mechanism or action.

The invention may provide a method for utilising the screen of a mobile telecommunication device to provide feedback to the device user. I.e. the user is notified via the screen when mixing is completed, when injection is completed, when device is powered on, when injection is initiated and other steps are followed.

In some embodiments, the invention may provide an “on-screen teaching method” which may replace the need for an instruction manual for use. Further, it may provide a method for automatically mixing pharmaceutical substances which are contained in dual chamber cartridge. The invention may provide a method for utilising the screen of a mobile telecommunication device to provide feedback to the device user. I.e. the user is notified via the screen when mixing is completed, when injection is completed, when device is powered on, when injection is initiated, and other measurable parameters or events in the injection process.

In some embodiments, the invention provides an injection pen system that may automatically be powered on when a primary drug container is inserted into the injection pen system.

### BRIEF DESCRIPTION OF DRAWINGS

FIG. 1 shows in planar view the main elements of an embodiment of a multi-use injection pen system according to the invention.

FIG. 2 shows in planar view an exploded embodiment of a multi-use injection pen according to the invention.

FIG. 3 shows in planar view the disengaged embodiments of a driver assembly and injection assembly of an injection pen system.

FIG. 4 shows in planar view an exploded embodiment of the driver assembly of an injection pen according to the invention.

FIG. 5 shows in planar view an embodiment of an exploded syringe assembly according to the invention.

FIG. 6 shows in planer view an embodiment of a dosage wheel of an injection assembly according to the invention.

FIG. 7 shows embodiments of primary drug containers which can be used with the invention.

FIG. 8 shows the drug container housing assembly of an embodiment of the invention.

FIG. 9 shoes the priming and trigger contact of an embodiment of the invention.

### ADVANTAGES OF THE INVENTION

The invention very advantageously provides a monitoring and guidance system for delivering substances by injection. Most advantageously, the invention system provides means to guide a user when to inject substances according to a predefined, but modifiable, program, such as for therapeutic drug injection, and monitors that the user has made the injection according to a treatment plan. Most advantageously, the program can be modified offline and remotely by a medical practitioner to best treat an indication using the invention by using the injection system's communication interface with a telecommunication device. The injection system obviates or minimises the need for a user manual to be consulted by a user self-injecting, particularly when commencing a program of injections needed only infrequently, say, once per week.

The invention advantageously provides a customisable modular system in which a much reduced number of parts have to be changed in order to use different kinds of primary drug containers in comparison with the prior art.

The injection system most advantageously obviates or minimises the need for a user manual to be consulted by a use self-injecting, particularly when commencing a program of injections needed only infrequently, say, once per week.

The invention advantageously provides a customisable modular system in which a much reduced number of parts have to be changed in order to use different kinds of primary drug containers in comparison with the prior art.

### Description of the Invention and Preferred Embodiments

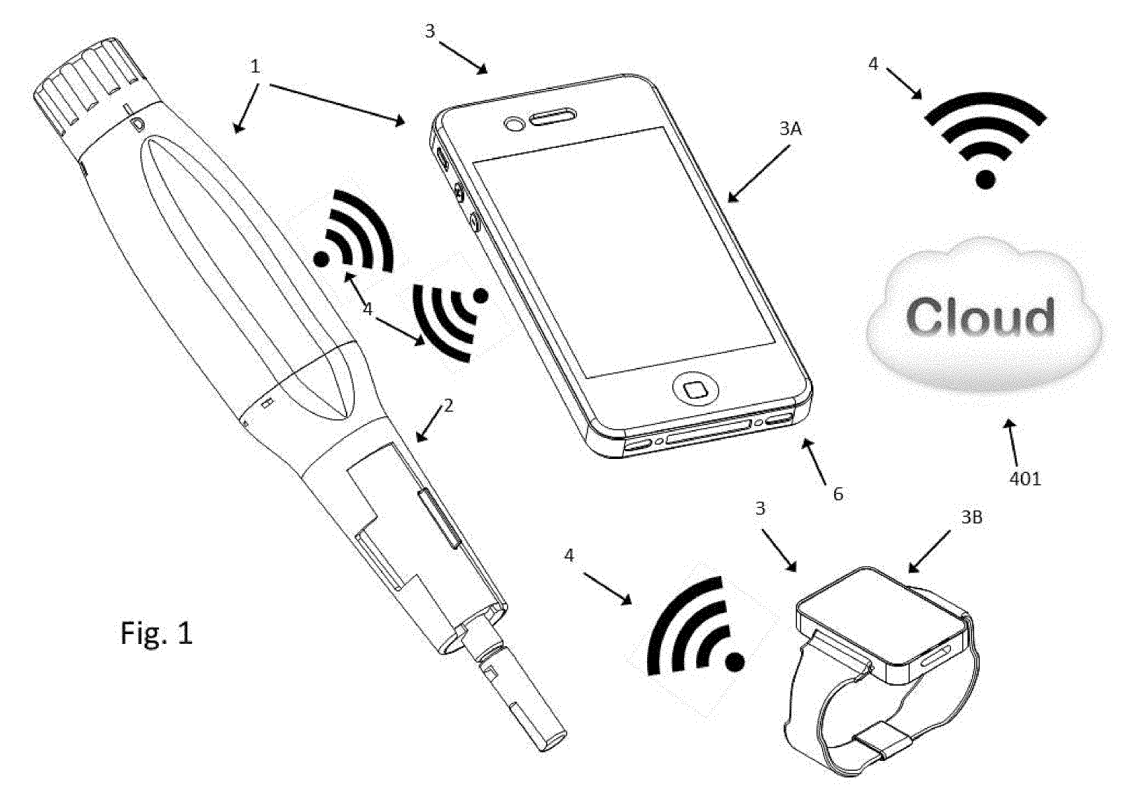

The invention is best understood by the description herein and associated drawings in the figures. It will be understood that the scope of the invention includes embodiments not shown in this disclosure and that the scope of the invention is limited only by the claims appended hereto. An embodiment of the invention is shown in FIG. 1, where an injection pen system 1 according to the invention incorporates an injection pen 2 incorporating a drug container, the injection pen suitable and configured for multiple uses including a first microprocessor, mobile communication device 3 including a second microprocessor 6 for transmitting and receiving data from the injection pen 2. The mobile communication device 3 may be any suitable device such as a mobile/cell phone. Alternatively, the mobile communication device may be a watch or other device that may be worn on the wrist, for example. Preferably the mobile communication device is a mobile/cell phone. Both the injection pen 2 and mobile communication device 3 may transmit and/or receive radio signals 4 by antennae for transmitting and receiving data. Preferably the antennae are disposed within the apparatus. The injection pen 2 incorporates a power source for powering its antenna, microprocessor, and other components requiring power. Power may be provided remotely, such as with radio waves. In the preferred embodiment, the mobile communication device incorporates a battery for a power source.

The invention includes a computer software program, which is preferably disposed in the telecommunication device 3, for interactively directing a user in carrying out an injection procedure, and collecting and storing data related to the procedure. The software (which may be firmware) integrated into the device is preferably designed to run and control all components integrated into the device. This may include components such as an electric actuator, dosage means, different delivery programs, wireless communication, device configuration, RAM, ROM, etc. The stored information may include, without limitation: the time, the place, injection volume, the injection site on the body, as well as information about the therapeutic substance in the primary drug container, such as the active compound, the manufacture date, the batch number, and the expiry date which may be programmed into a device internal chipset. The computer software, which is preferably disposed on the telecommunication device, may have an interface to other health-related computer software applications. Other data may be collected in some embodiments. This data is embodied in the signals transmitted by the antennae and automatically captured by the communication between the injection pen 2 and the software application. Further, an advantage of loading the software on a mobile communication device 3 is that the data can be transmitted to various recipients, such as of the practitioner prescribing the therapeutic treatment, the manufacturer of the medicament, or the controller of a clinical trial. The mobile phone application allows interaction with a user database of injections, frequency, and therapeutic treatment substances, concentrations, and the like.

The invention also provides a mechanism to automatically prime (expel air) from the primary drug container and provide instance feedback to the user after priming is completed via the GUI of the mobile communication device.

Injection System

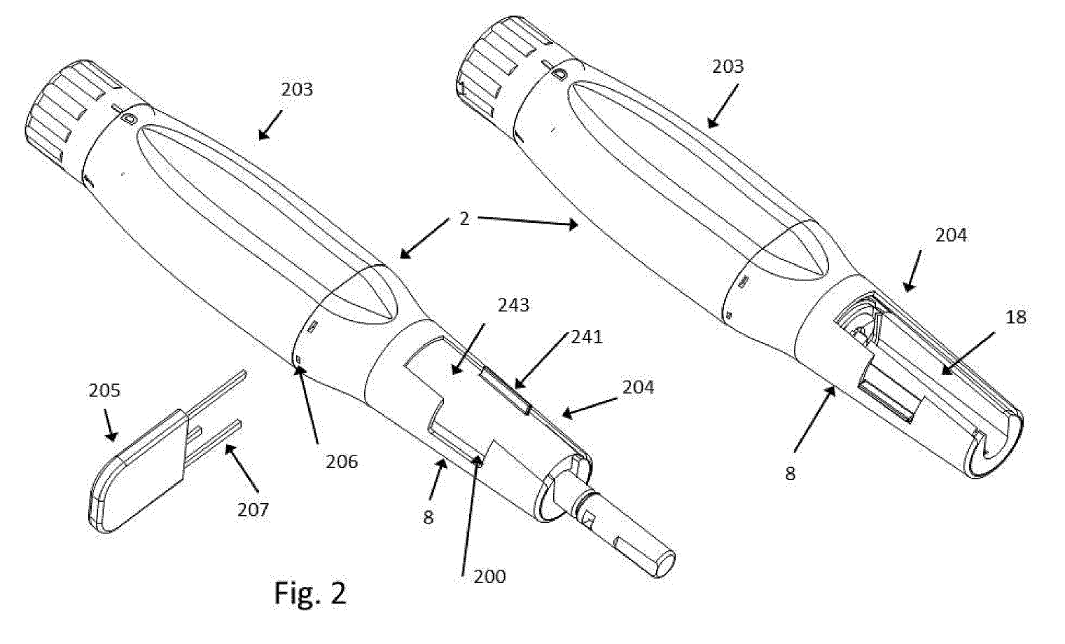

FIG. 1 shows an embodiment of elements of a system and apparatus for guiding and monitoring the injection of fluids using an injection pen incorporating a primary drug container. The embodiment incorporates an injection pen delivery device 2 in communication with a mobile communication device 3, which, in turn, may be in communication with databases and stakeholders in providing diagnosis and health benefits 401 to the user of the injection system. Most advantageously, the delivery device comprises of an injection pen for primary drug containers, preferably a pre-filled syringe and communication means which exploits the speed and bandwidth of the internet to acquire, store, and access data and information. FIG. 1 shows that the mobile communication device 3 may take any convenient form such as a mobile telephone 3A or a watch 3B. The injection system may be complimented with an external charging device to recharge the power source of the injection pen located in the driver assembly 203 (shown in FIG. 2). Most advantageously the injection system can be used in combination with the computer software disposed in a telecommunication device or as a stand-alone device without using a telecommunication device. Embodiments of the invention using the injection pen without the software disposed in the telecommunication device may include the injection assembly 1 internally storing injection data and transmitting such data to the telecommunication device when a connection is established between injection assembly 1 and telecommunication device 3.

The features of an embodiment of an injection pen 2 are shown in in FIG. 2. FIG. 2 shows the assembled injection pen 2 comprises of two main assemblies, i.e., a driver assembly 203 and a syringe assembly 204. The driver assembly 203 and syringe assembly 204 may be engaged/disengaged with a locking key 205 having pins 207 for engagement with engagement holes 206. The syringe assembly 204 includes a primary drug container housing 239. The syringe assembly 204 includes a housing door 200 which is shown in the open position, revealing the cavity 243 for the drug container. The housing door 200 is used to lock the primary container in place when the housing door 200 is closed.

The injection pen is designed in a way that only a minimal number of parts need to be changed for using a different primary drug container. Advantageously the syringe assembly includes identification so that the firmware of the driver assembly 203 may recognise the kind of primary drug container used in the syringe assembly 204. The injection pen is preferably designed in an ergonomic way.

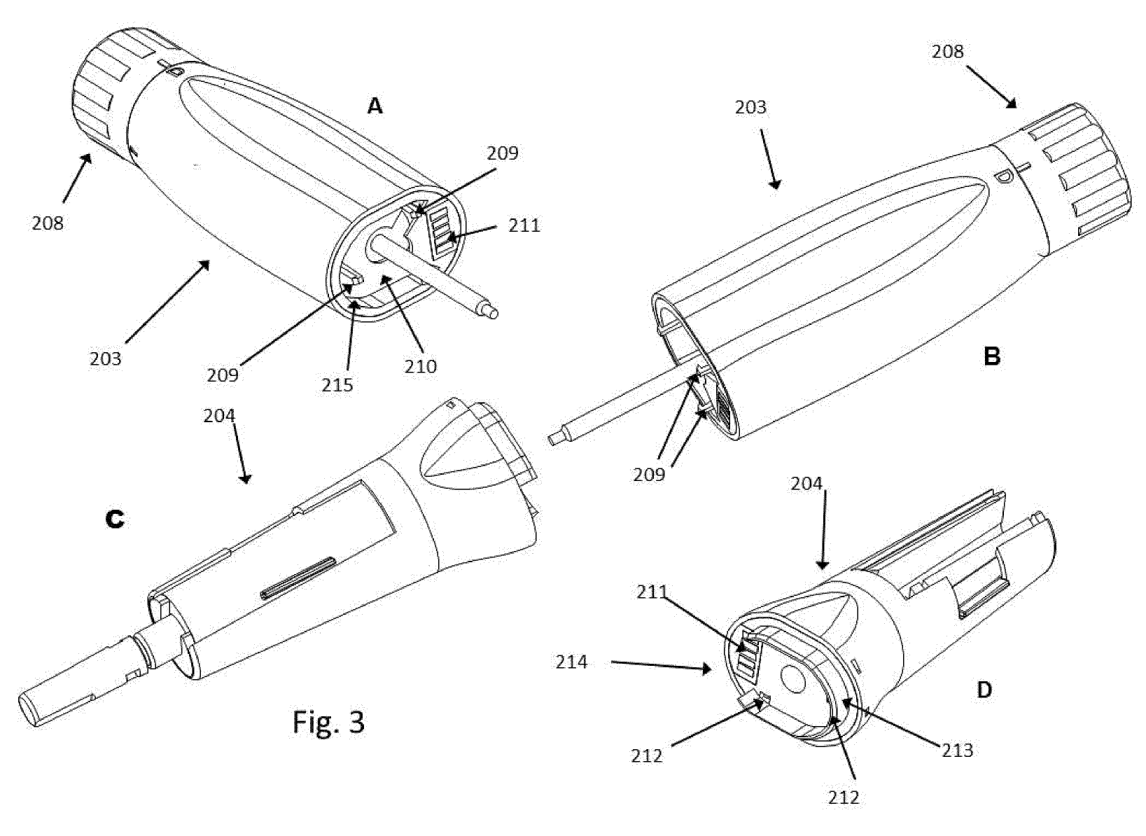

FIG. 3 shows embodiments of the driver assembly 203 in two orientations A, B and the syringe assembly 204 in two orientations C, D when the assemblies are disengaged. Preferably, the driver assembly 203 and syringe assembly 204 engage securely with engagement means. The engagement means may be in the form of clips 209 for locking into appropriately figured cut-outs 212 as shown from different perspectives in FIG. 3 A,B. The engagement may be facilitated by embodiments incorporating male 214 and female 215 configurations of the engaging ends of the driver assembly 203 and injection assembly 204 as illustrated in FIG. 3A where the driver assembly 203 incorporates a recess 210 to accommodate a projection 213 on the syringe assembly 204 FIG. 3 D, configured as a male end 214 which slides into the recess 210 of the female end 215 driver assembly. When the driver assembly 203 and syringe assembly 204 are fully engaged, clips 209 engage the cut-outs 212.

Sliding the male end 214 of the syringe assembly 204 into the female end 215 of the driver assembly 203 conveniently engages the SIM contacts 211 of each of the driver assembly 203 and injection assembly 204 allowing electrical conduction between the two assemblies, in particular, the signals for monitoring the process of injection.

FIG. 3B also shows the driver assembly which may contain means for controlling the amount of fluid delivered from the primary drug container 208. Preferably the means is a dosage wheel.

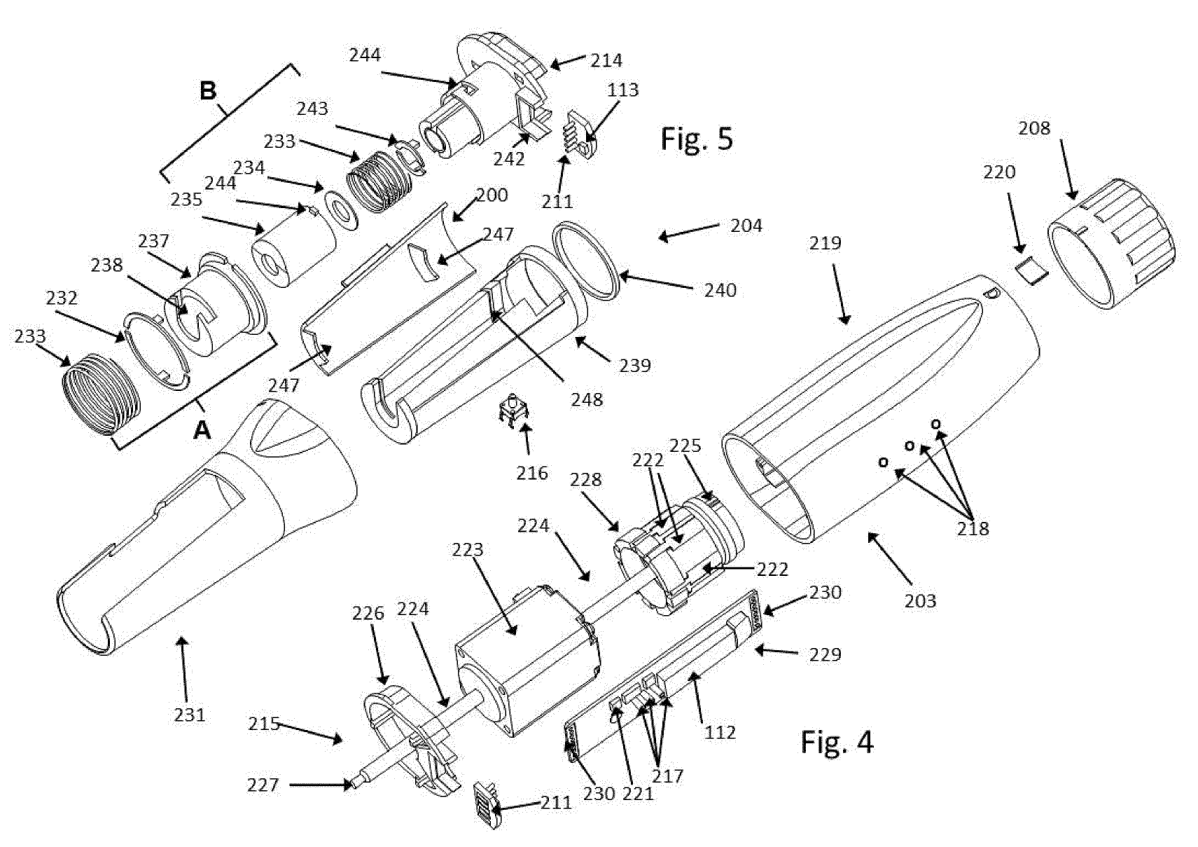

FIG. 4 shows an exploded embodiment of a driver assembly 203 of a multiple use injection pen system. The driver assembly preferably includes a dosage wheel 208 for determining the amount of fluid to be injected using the injection pen system. Inside the dosage wheel 208 may be a metal contact 220 for conducting electrical signals when the dosage wheel 208 is rotated to different positions. The dosage wheel 208 preferably engages one end of the driver assembly housing 219 which may house an actuator with lead screw 224, dosage shaft 228, electric actuator 223, and an end piece 226. The end piece 226 preferably takes a female configuration 215 as described above. The lead screw 224 includes an end portion 227 configured for engaging and moving the piston of a syringe or other drug container in the syringe assembly 204 portion of the injection pen system 1. The dosage shaft 228 incorporates electrical contacts 222 which make electrical contact with the metal contact 220 inside the dosage wheel 208 as the dosage wheel is rotated.

The driver assembly housing 219 preferably includes space for a main circuit board 229 preferably including several components such as microprocessor, wireless module for acquiring and transmitting signals as the injection process is carried out. The main circuit board 229 preferably incorporates a rechargeable or replaceable battery 112 for energising the electrical circuit for generating signals. The battery 112 also powers the microprocessor 221 configured with computer software for generating and acquiring data related to the injection process. Preferably, the main circuit board incorporates light-emitting diodes (LEDs) 217 or other low energy indicators for indicating the steps of the injection process. Preferably, embodiments incorporating LEDs also incorporate windows or other apertures 218 to allow light transmission for indicating the status of the LEDs. The main circuit board 229 also includes electrical contacts for engaging the SIM contacts 211 of the female end 215 of the driver assembly. Further, it includes a microprocessor 221 and wireless module. The micro-processor is used to drive and control the entire system, including driving and controlling an actuator. The main board also includes storage to store different delivery programs.

FIG. 5 shows an exploded embodiment of a syringe assembly 204 of a multiple use injection pen system, the syringe assembly having at one end a male portion 214 for engaging the driver assembly 203 and front housing 231 at the other end. Within the front housing 231 of the syringe assembly 204 is a drug container housing 239 comprising of a housing door 200 for covering a prefilled syringe or other drug container for delivering a drug using the injection pen system. The male end portion 214 incorporates a holder 242 for holding a SIM contact 211 and identification element 113 in any suitable form such as ROM, resistor or other suitable component. The SIM contact is in electrical communication with the corresponding SIM contact of the driver assembly 203 when the injection pen system is ready for use.

The injection system's main board 229 is powered on when a primary drug container is inserted into the container housing 239 by activating a switch, preferably a push button switch 216, which is in electrical communication with the main board 229 via SIM contacts 211.

The flanges 254 of the plunger of the drug container 253, such as a syringe, engage with the syringe holder 237 by slotting into a cut-out 238. The syringe holder 237 is biased towards the ends of the assembly by springs 233. Detaching the cap from the primary drug container or detaching on the primary drug container generates a signal to commence priming. The signal is generated by making contact with priming contact 232 which is disposed on housing 237 and priming contact ring 246 which is preferably press-fitted into cover housing 239. Preferably, priming contact 232 is spaced adequately from priming contact base ring 246 and makes contact when the primary drug container is moved towards the front housing 231 by either removing a safety cap from the primary drug container or pulling on the primary drug container towards the front housing 231.

The syringe assembly is primed for injection of a drug-containing liquid when a drug container (not shown) is placed within drug container housing 239.

A second contact assembly is the trigger contact B. The trigger contact assembly B consists of trigger housing 236, contact base ring 234, spring element 233 and trigger contact 243.

A contact base ring 234 is spaced from trigger contact 243 with spring element 233 in a default position. The trigger housing is kept in its default position by trigger housing lug 244 which engages with a cut-out 245. When the drug primary drug container is pressed against the skin, contact base ring 234, which is fitted into trigger housing 236, is pushed onto the trigger contact 234 by a minimal axial movement of the primary drug container towards the driver assembly 203.

Housing door 200 is housed in drug container housing 239 and kept in axial position by door rib 247 which engages with cut-out 248 located in container housing 239. Radially it is kept in place by being sandwiched between container housing 239 and front housing 231 when container housing 239 is assembled with front housing 231. Housing door 200 is conveniently rotationally moveable between opened and closed positions.

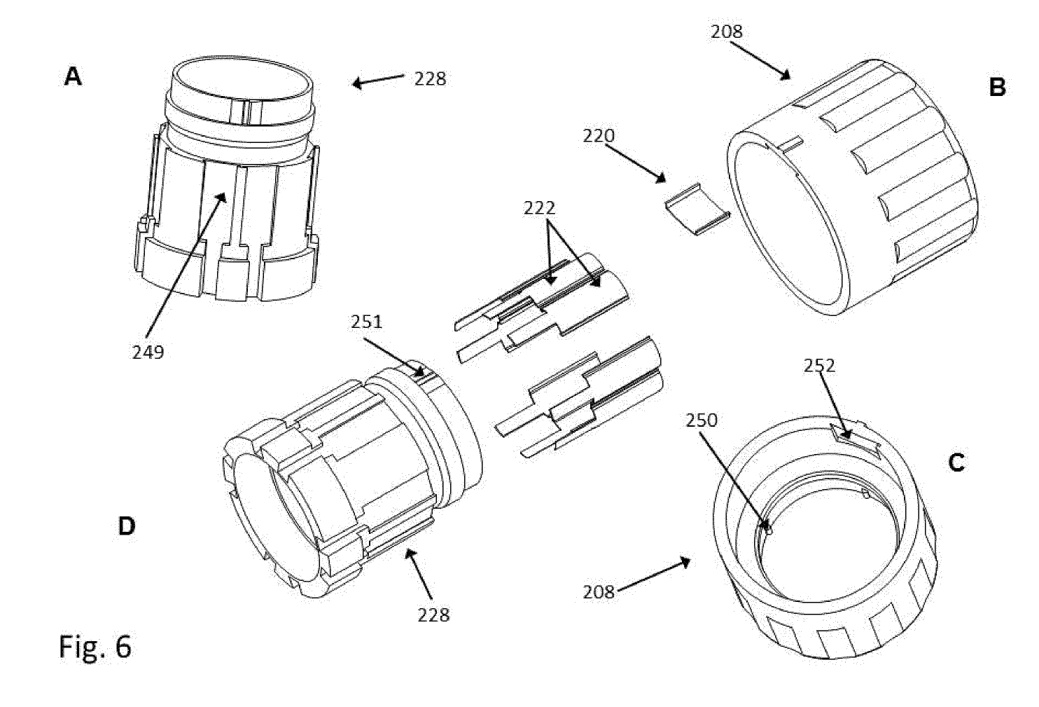

FIG. 6 shows the dosage wheel in exploded view, consisting of dosage shaft 228 which is inserted in driver housing 219, electrical contacts 222, contact 220 affixed in slot 252 of dosage wheel 208 and dosage wheel 208. The embodiment shown in FIG. 6 shows four pairs of electrical contacts 222, which are inserted into slot 249 of dosage shaft 228. The four pairs of electrical contacts 222 provide a total of four different contact possibilities and are triggered when contact 220 bridges two electrical contacts 222. It's understood that the number of paired electrical contacts 222 can vary depending on the required dosage volumes. Alternatively, the contacts may be replaced with a micro-positioning sensor known in the art. The array of electrical contacts is ideally used to trigger different computer software sub-routines stored on the main board. Advantageously the dosage wheel may include a mechanical feedback option in order to use the system when in communication with a telecommunication device. Rib 250 engages with slot 251 when dosage wheel 208 is rotated and a position is reached where contact 220 bridges electrical contacts 222.

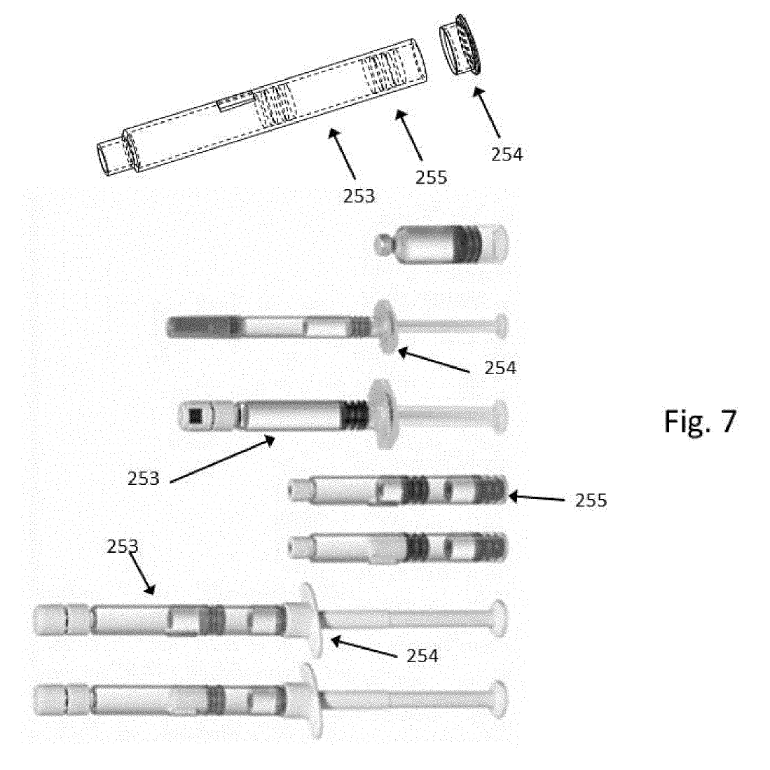

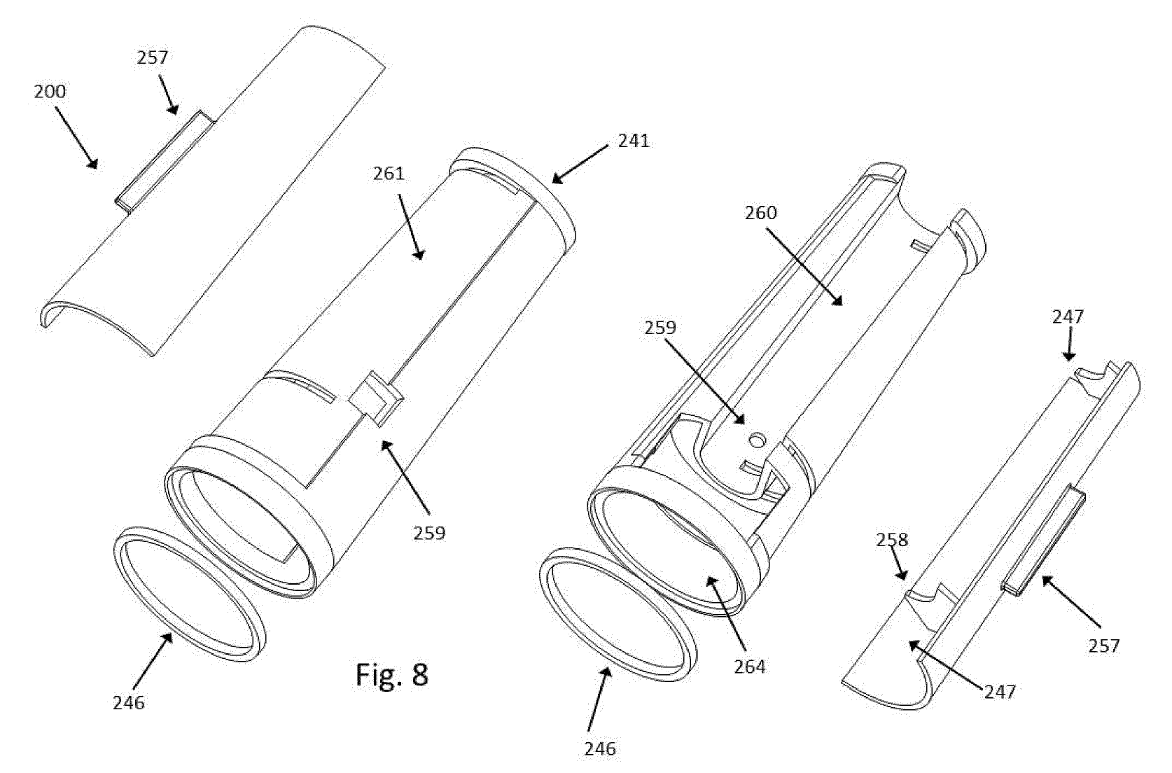

The invention most advantageously may be used with many configurations of primary drug containers. FIG. 7 shows a range of different primary drug containers 253 known in the art which may be used with the invention described herein. A primary drug container for use with the invention may comprise of flange 254, piston 255 and cap 256.

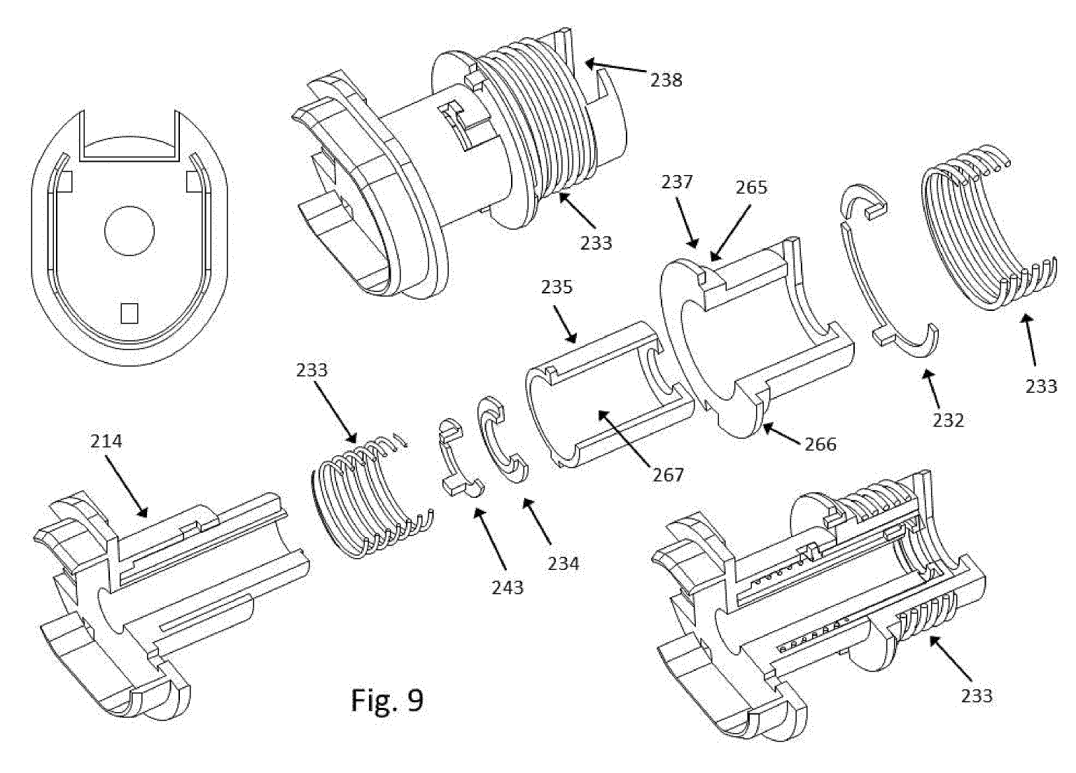

FIG. 8 shows a sub-assembly of the syringe assembly 204, consisting of drug container housing 241, housing door 200, and priming contact base ring 246. Housing door 200 may incorporate a finger grip 257 which can be used to open and close the door. Door 200 advantageously may incorporate door ribs 247 which engage with cut-out 248 and stop the door 200 from moving axially when inserted in drug container housing 241. Face 258 of rib 247 keeps the primary drug container 253 in place when housing door 200 is closed. Advantageously, the housing 241 features cut-outs 259 to receive a push-on switch 216 known in the art which enables the device describe herein to power on when drug container 253 is inserted in cavity 260. Preferably drug container housing 24 holds a cut-out 261 for receiving housing door 200. Recess 261 provides a nest for holding the housing door 200 when the sub-assembly described in FIG. 9 is inserted into syringe assembly 204. Opening 262 provides means to insert a flange 254 of primary drug container 253.

FIG. 9 shows the priming/mixing contact assembly A and trigger contact B in an exploded view with a sectional cut-out, isometric view and isometric view with a sectional cut-out. Spring 233 is sandwiched between the front face 264 of housing 241 and front face 265 of syringe holder 237, biasing the syringe holder 237 towards the driver assembly 203. Contact ring 246 is inserted onto front face 263 of drug container housing 241.

Priming contact 232 is disposed on front face of syringe holder 237 and contacts with contact ring 246 when the cap 256 of primary drug container 253 is removed. Removing of the cap 256 will pull the drug container 253 towards the syringe assembly 204. Inside the syringe holder 237 is a trigger housing 236 which is biased towards the syringe assembly 204 by a spring element 233. Contact base ring 234, which is affixed to front face 267 of trigger housing 235, makes contact with trigger contact 243 when the device is contacts the skin of a user and the drug container 253 is minimally moved towards the driver assembly 203. Trigger contact is affixed to front face 269 of male end of syringe assembly. In the device default position, when the device is powered-up, base ring 234 and trigger contact 243 as well as priming contact 232 and contact ring 246 are minimally spaced apart in a way that no contact is made. Preferably, trigger housing 235 is kept in place by a lug 244 which engages with cut-out 244.

Computer Software

The computer software incorporated into the invention includes many different embodiments for monitoring and controlling the drug injection process, including generating and storing data associated with the drug and the process. Most advantageously, the multi-use system includes embodiments which can keep longitudinal data over multiple injections over time. In one embodiment, the computer software for monitoring and controlling the operation of an injection includes the initial steps for the injection system. In an initial step 300 the primary drug container is inserted into the cover housing 239 when the housing door 200 is in the open position. Inserting the primary drug container 301 will power the device by activating the push-on power switch 216. At this point in time a signal 304 may be transmitted to the application via the wireless module. The firmware, process 302, may check the device configuration and check which kind of syringe assembly 204 is assembled with diver assembly 203. Preferably, the different device configurations are internally stored 305 and the appropriate device configuration may be loaded as part of process 306 and 307. Process 308 is device ready status. At this point in time another signal 309 may be transmitted to the computer software application. Process 311 is triggered by the priming/mixing switch shown in FIG. 5. Activating the priming/mixing switch by removing the cap or pulling on the primary drug container initiates the priming or mixing process 312 and the actuator is moved to a predetermined position within process 313 until priming mixing is completed 314. At this point in time another signal 315 may be transmitted to the computer software application. If a dosage wheel is incorporated into the device, process 317 allows the end-user to select the right dose via dosage wheel 208. At this point in time another signal may be send to the app 316. An accelerometer may be used within process 319 to record the shaking of the device in order to fully mix the drug. Once this process is completed, process 320 renders the device ready for injection. At this point in time another signal 322 may be send to the app triggering certain event within the app. Process 325 is a manual process executed by the end-user. Pushing the device onto the skin will initiate process 326 which is triggered by activation the trigger switch. At this point in time another signal 324 may be transmitted to the application. The actuator is moved to a predefined position as part of process 327 until the dose is fully delivered 328. At this point in time another signal 329 may be transmitted to the application. The injection data may be device internally stored in process 331. Process 332 is a manual process in which the end-user is asked to remove the primary drug container. At this point in time another signal 335 may be send to the application. The device is automatically powered off 334 when primary dug container is removed and push-on switch 216 is deactivated.

Computer Software (Mobile Application)

The invention includes a first microprocessor 6 programmed with computer software preferably disposed within a telecommunication device, to initiate and control the transmission of communication waves between the injection pen 2 and the mobile communication device 3. Modern mobile/cell telephones conveniently operate by computer software integrated into their operating systems. The invention includes software programs integrated into the operating system platform of a mobile/cell phone to transmit and receive data from the operation of the injection pen 2. Preferably, the telecommunication device will receive multiple signals, 304, 309, 315, 310, 321, 322, 326, 324, 329, 330 and 335 from the injection pen 2. These signals may trigger different events or sub-routines within the computer software (app). Preferably, the computer software receives and stores signals from the injection pen indicating the status and progress of the injection process, including, for example, priming of the primary drug container, starting the injection of fluid into a subject, ending of the injection process, time of the injection process, and other relevant information. The computer software may include sub-routines which prompt a user to make an injection. The prompt may include an audible signal to alert the user to commence an injection or alternatively the user may receive a message via the mobile devices' messenger system. The injection software may be downloadable from the Internet onto the mobile communication device 3. The injection may be modifiable and updatable to include changes in the aspects such as the timing of prompts to a user to make an injection. Preferable to computer software provides a routine to record injection locations so that changing injecting location can be carried out more accurately. Preferably, the algorithm is programmed in a manner that subsequent signal or data packages received from the pen trigger different prompts and message within the application. Preferably, the software is programmed enabling the patient to use the device without needing a printed instruction for use. The software provides feedback forms enabling the user to enter user feedback at any given time. The software may have different predefined modes allowing the user to select an appropriate mode depending on his preference and experience with the injection system. In example, in the beginning of a treatment the user may wish to be guided trough step by step whereas once the user is familiar with the system he may wish changing to a simpler mode, only using certain functions, in example disabling feedback forms and prompts etc.

I claim:

1. An injection pen system comprising of: a driver assembly for a drug container; and a syringe assembly; a power source for generating electrical energy; electrical communication means in each of the driver assembly and syringe assembly wherein the driver assembly and syringe assembly are in electrical communication; and at least one microprocessor programmed to monitor electrical signals produced by the injection pen components.

2. The injection pen system of claim 1 when the electrical signals are produced at predefined intervals.

3. The injection pen system of claim 1 wherein the electrical signals are wirelessly transmitted.

4. The injection pen system of claim 1 where the transmitted signals trigger commands within a computer program monitoring the injection process.

5. The injection pen system of claim 1 wherein said programmed microprocessor is spaced from said driver assembly and syringe assembly.

6. The injection pen system of claim 1 further incorporating a two-way switch.

7. The injection pen system of claim 6 wherein said switch is activated by a trigger.

8. The injection pen system of claim 6 further comprising of a primary drug container having a cap wherein the trigger activation occur by removing said cap.

9. The injection pen system of claim 7 further comprising of a primary drug container wherein said trigger is activated by pushing said primary drug container adjacent a subject.

10. The injection pen system of claim 8 wherein said activation causes mixing of a lyophilised substrate with a fluid.

11. The injection pen system of claim 9 wherein said activation causes mixing of a lyophilised substrate with a fluid
